(19) United States
(12) Patent Application Publication
(10) Pub. No.: US 2019/0152093 A1
(43) Pub. Date: May 23, 2019

(54) METHODS FOR IMPROVED COPPER PENETRATION IN WOOD

(71) Applicant: Koppers Performance Chemicals Inc., Pittsburgh, PA (US)

(72) Inventors: Jun Zhang, Peachtree City, GA (US); Peter Tham, Morrow, GA (US); Barry Sewell, Covington, GA (US); Jacob T. McBrayer, Locust Grove, GA (US); John Horton, Peachtree City, GA (US)

(21) Appl. No.: 16/192,421

(22) Filed: Nov. 15, 2018

Related U.S. Application Data
(62) Division of application No. 15/335,787, filed on Oct. 27, 2016.
(60) Provisional application No. 62/353,711, filed on Jun. 23, 2016.
(60) Provisional application No. 62/248,444, filed on Oct. 30, 2015.

(51) Int. Cl. B27K 3/52; C07C 215/40; A01N 33/12; C07C 211/63; B27K 3/34; A01N 25/30; B27K 3/22; A01N 59/20

(52) U.S. Cl. CPC B27K 3/52; C07C 215/40; A01N 33/12; C07C 211/63; B27K 2240/20; A01N 25/30; B27K 3/22; A01N 59/20; B27K 3/34; B27K 3/343

(57) ABSTRACT

The present invention is directed to amine soluble, or solid copper triazole based wood preservative formulations containing certain types of quaternary ammonium compounds, or nonionic sufactants, and the use of these quaternary ammonium compound-, or nonionic surfactant-containing formulations to pressure treat and preserve wood. The addition of the quaternary ammonium compounds, or nonionic surfactants to the wood preservation treatment solutions allows improved penetration of the copper from the solution into wood and reduces the duration of time required to effectively pressure treat the wood with the preservation composition.

### CROSS-REFERENCE TO RELATED APPLICATIONS

This application is a divisional of U.S. application Ser. No. 15/335,787, filed Oct. 27, 2016, which claims the benefit under 35 U.S.C. § 119(e) of U.S. Provisional Patent Application No. 62/353,711, filed Jun. 23, 2016, and U.S. Provisional Patent Application No. 62/248,444, filed Oct. 30, 2015, each of which are herein incorporated by reference in their entirety.

### FIELD OF THE INVENTION

The present invention relates to methods of enhancing the penetration of copper into wood products by the addition of quaternary ammonium compounds, or nonionic surfactants, to copper plus triazole based preservative formulations, methods of using such formulations to treat wood, and wood products treated using the formulations and the methods.

### BACKGROUND OF THE INVENTION

Copper plus triazole based wood preservative formulations are commonly used to pressure treat and preserve wood. The copper present in these formulations can be either solubilized copper solutions or dispersed particles of copper compounds/copper complexes and the copper or copper compound, acts as a biocide, fungicide, and insecticide, protecting wood pressure treated with the copper compounds against rot and decay caused by fungal, bacterial, and insect infestation.

However, when such formulations are used to pressure treat varieties of wood that are relatively difficult-to-treat (e.g., Douglas fir, Hem-Fir, some southern yellow pines, certain red pine or ponderosa pine, and other refractory wood species), the penetration of the copper into the wood may be limited and may not meet relevant copper penetration conformance standards; for example, the American Wood Protection Association Standard T1-15 “Use Category System: Processing and Treatment Standard” (2015) which is incorporated herein by reference in its entirety. Moreover, the processing time required to treat these varieties of wood with such copper plus triazole wood formulations is increased in comparison with other traditional systems.

As a result, there remains a need for solubilized copper or dispersed solid copper, plus triazole formulations that can effectively and timely pressure treat these and other varieties of refractory woods.

### SUMMARY OF THE INVENTION

The present invention is directed, in certain embodiments, to methods for increasing copper penetration of a wood preservative composition into a wood product and/or decreasing the time required to achieve proper penetration of a wood preservative composition into a wood product, the method comprising contacting a wood preservative composition with a wood product, wherein said wood preservative composition comprises: (a) a solubilized copper compound, or a solid copper compound; (b) a triazole; and (c) a quaternary ammonium compound, or a nonionic surfactant; wherein said wood preservative composition penetrates said wood product to a greater degree than said wood preservative composition lacking a quaternary ammonium compound, or a nonionic surfactant.

In certain embodiments of the invention, the quaternary ammonium compound has a chemical structure comprising:

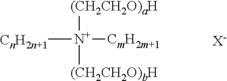

wherein the value of m is at least 1 and at most 20, the value of n is at least 1 and at most 20, the value of a is at least 1 and at most 5, the value of b is at least 1 and at most 5, and X⁻ is an anion selected from the group consisting of borate, chloride, carbonate, bicarbonate, bromide, iodides, formate, acetate, propionate, and other alkyl carboxylates. In certain embodiments, the value of m, n is 10 or 12, the value of a is 1, the value of b is 1, and X⁻ is borate, chloride, propionate, carbonate, or bicarbonate.

In certain embodiments of the invention, the quaternary ammonium compound has a chemical structure comprising:

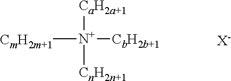

wherein the value of m is at least 1 and at most 20, the value of n is at least 1 and at most 20, the value of a is at least 1 and at most 5, the value of b is at least 1 and at most 5, and X⁻ is an anion selected from the group consisting of borate, chloride, carbonate, bicarbonate, bromide, iodide, formate, acetate, propionate, acetate, propionate, and other alkyl carboxylates. In certain embodiments, the value of in is at least 8 and at most 14, and the value of n is at least 8 and at most 14. In certain embodiments, the value of m is 10 or 12, the value of n is 10 or 12, the value of a is 1, the value of b is 1. In certain embodiments X⁻ is borate, chloride, propionate, carbonate, or bicarbonate. In certain embodiments, the value of in is 10 and the value of n is 10. In certain embodiments, the value of m is 12 and the value of n is 12.

In certain embodiments of the invention, the quaternary ammonium compound has a chemical structure comprising:

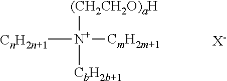

wherein the value of m is at least 1 and at most 20, the value of n is at least 1 and at most 20, the value of a is at least 1 and at most 5, the value of b is at least 1 and at most 5, and X⁻ is an anion selected from the group consisting of borate, chloride, carbonate, bicarbonate, bromide, iodide, formate, acetate, propionate, acetate, propionate, and other alkyl carboxylates. In certain embodiments, the value of m is at least 8 and at most 14. In certain embodiments, the value of n is at least 8 and at most 14. In certain embodiments, the value of a is 1, the value of b is 1. In certain embodiments X⁻ is borate, chloride, propionate, carbonate, or bicarbonate. In certain embodiments, the value of m is 10 and the value of n is 10. In certain embodiments, the value of m is 12 and the value of n is 12.

In certain embodiments of the invention, the nonionic surfactant compound is selected from the group consisting of aromatic ethoxylates, alkylphenol ethoxylates, such as octylphenol ethoxylates, nonylphenol ethoxylates, dinonylphenol ethoxylates, phenol ethoxylates and dodecylphenol ethoxylates.

In certain embodiments of the invention, the nonionic surfactant compound is an alcohol ethoxylate. The alcohol may be a primary or a secondary alcohol. The alcohol may be branched or linear or mixed branched and linear.

In certain embodiments of the invention, the nonionic surfactant compound is a copolymer of ethylene oxide (EO) and propylene oxide (PO), or the product of the ethoxylation of alcohols or phenols with EO or PO copolymer.

In certain embodiments of the invention, the nonionic surfactant compound is a fatty amide, an alkanolamide or an ethylene bisamide.

In certain embodiments of the invention, the nonionic surfactant compound is a nonionic ester, such as a fatty acid ester, a glycerol ester, a glycol ester, an alcohol ester, an ethoxylated fatty acid, glycol and polyethylene glycol (PEG) esters, ethoxylated fatty oils.

In certain embodiments, the solubilized copper compound is prepared from cuprous oxide, cupric oxide, copper hydroxide, copper carbonate, basic copper carbonate, copper oxychloride, copper metal, or copper borate; and a solubilizing agent. In certain embodiments, the solubilizing agent is an alkanolamine, such as, for example, monoethanolamine, ethanolamine, diethanolamine, triethanolamine or ammonia, and combinations thereof.

In certain embodiments, the solid copper compound is prepared from cuprous oxide, cupric oxide, copper hydroxide, copper carbonate, basic copper carbonate, copper oxychloride, copper metal, or copper borate, and a dispersant or an emulsifier.

In certain embodiments, the triazole is epoxiconazole, triadimenol, propiconazole, prothioconazole, metconazole, cyproconazole, tebuconazole, penflufen, flusilazole, paclobutrazol, fluconazole, isavuconazole, itraconazole, voriconazole, pramiconazole, ravuconazole, or posaconazole.

In certain embodiments, the quaternary ammonium compound is present in the wood preservative treating composition in an amount between about 0.01% (wt/wt) to about 0.5% (wt/wt); or between about 0.01% (wt/wt) to about 0.2% (wt/wt); or between about 0.03% (wt/wt) to about 0.15% (wt/wt); or between about 0.05% (wt/wt) to about 0.10% (wt/wt); or between about 0.1% (wt/wt) to about 0.2% (wt/wt).

In certain embodiments, the nonionic surfactant is present in the wood preservative treating composition in an amount between about 0.01% (wt/wt) to about 5% (wt/wt); or between about 0.01% (wt/wt) to about 1.0% (wt/wt); or between about 0.05% (wt/wt) to about 0.25% (wt/wt); or between about 0.05% (wt/wt) to about 0.15% (wt/wt); or between about 0.05% (wt/wt) to about 0.1% (wt/wt).

In certain embodiments, the nonionic surfactant in the wood preservative treating composition has an HLB value greater than 10, 11, 12, 13, 14, or 15; or an HLB value between 10 and 40; or between 10 and 30; or between 10 and 20; or between 14 and 18. As used herein, the hydrophilic-lipophilic balance (HLB) of a surfactant is a measure of the degree to which it is hydrophilic or lipophilic, determined by calculating values for the different regions of the molecule, as described by Griffin in 1949 and 1954. Other methods have been suggested, notably in 1957 by Davies. See Griffin, William C. (1949), “Classification of Surface-Active Agents by ‘HLB’” (PDF), Journal of the Society of Cosmetic Chemists 1 (5): 311-26; Griffin, William C. (1954), “Calculation of HLB Values of Non-Ionic Surfactants” (PDF), Journal of the Society of Cosmetic Chemists 5 (4): 249-56; and Davies J T (1957), “A quantitative kinetic theory of emulsion type, I. Physical chemistry of the emulsifying agent” (PDF), Gas/Liquid and Liquid/Liquid Interface (Proceedings of the International Congress of Surface Activity), pp. 426-38, each of which is hereby incorporated herein by reference in their entireties.

In certain embodiments, the total copper azole concentration is present in the wood preservative treating composition in an amount between about 0.01% (wt/wt) to about 5.0% (wt/wt); or between about 0.05% (wt/wt) to about 2.0% (wt/wt); or between about 0.1% (wt/wt) to about 1.0% (wt/wt); or between about 0.2% (wt/wt) to about 0.8% (wt/wt); or between about 0.5% (wt/wt) to about 1.5% (wt/wt).

In certain embodiments, copper penetration passing rate in the wood product contacted with said wood preservative composition is at least about 5% greater; or at least about 10% greater; or at least about 15% greater; or at least about 20% greater; or at least about 25% greater; or at least about 30% greater; or at least about 35% greater; or at least about 40% greater; or at least about 45% greater; or at least about 50% greater; or at least about 55% greater; or at least about 60% greater; or at least about 65% greater; at least about 70% greater; at least about 75% greater; at least about 80% greater than the copper penetration passing rate of said wood product treated with said wood preservative composition lacking said quaternary ammonium compound, or said nonionic surfactant.

In certain embodiments, contacting the wood preservative composition with a wood product comprises the step of applying a pressure of between about 50 psi to about 200 psi to said wood product and said wood preservative composition. In certain embodiments, the pressure is between about 100 psi to about 150 psi.

In certain embodiments, the wood product is contacted with the wood preservative composition for at least about 1 minute to at least about 300 minutes. In certain embodiments the contacting is done for at least about 10 minutes to at least about 120 minutes. In certain embodiments the contacting is done for at least about 30 minutes to at least about 90 minutes. In certain embodiments the contacting is done for at least about 90 minutes to at least about 240 minutes.

In certain embodiments, the wood product is a sawn product such as a salable wood product. In certain embodiments, the wood product is lumber.

In certain embodiments, the wood product is a wood species selected from the group consisting of Douglas fir, Hem-fir, Nordic pine, Scotts pine, Norway spruce, Sitka spruce, southern yellow pine, incised Douglas fir, incised Hem-fir, Spruce pine fir, red pine, and ponderosa pine.

In certain embodiments, the invention is also directed wood products that have been treated using the methods disclosed herein.

### DETAILED DESCRIPTION OF THE INVENTION

The present invention is directed to solubilized copper, or solid copper, plus triazole preservation compositions to which certain quaternary ammonium compounds (also known as “quats”), or a nonionic surfactant, have been added, as well as to methods of preserving wood by pressure treating woods with such compounds. It has been discovered that the addition of these certain quaternary ammonium compounds, or nonionic surfactants, (collectively referred to as “penetration enhancers”) can improve the penetration of the copper compounds into wood, as well as reduce the time required to effectively pressure treat the wood with the preservation composition. The penetration enhancers are quaternary ammonium compounds or nonionic surfactants, as described in detail below.

In certain embodiments of the invention, the quaternary ammonium compound has a chemical structure comprising:

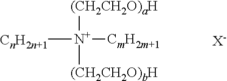

wherein the value of m is at least 1 and at most 20, the value of n is at least 1 and at most 20, the value of a is at least 1 and at most 5, the value of b is at least 1 and at most 5, and X⁻ is an anion selected from the group consisting of borate, chloride, carbonate, bicarbonate, bromide, iodides, formate, acetate, propionate, and other alkyl carboxylates. In certain embodiments, the value of m, n is 10 or 12, the value of a is 1, the value of b is 1, and X⁻ is borate, chloride, propionate, carbonate, or bicarbonate.

In certain embodiments of the invention, the quaternary ammonium compound has a chemical structure comprising:

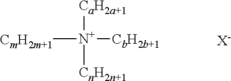

wherein the value of m is at least 1 and at most 20, the value of n is at least 1 and at most 20, the value of a is at least 1 and at most 5, the value of b is at least 1 and at most 5, and X⁻ is an anion selected from the group consisting of borate, chloride, carbonate, bicarbonate, bromide, iodide, formate, acetate, propionate, acetate, propionate, and other alkyl carboxylates. In certain embodiments, the value of m is at least 8 and at most 14, and the value of n is at least 8 and at most 14. In certain embodiments, the value of m is 10 or 12, the value of n is 10 or 12, the value of a is 1, the value of b is 1. In certain embodiments X⁻ is borate, chloride, propionate, carbonate, or bicarbonate. In certain embodiments, the value of m is 10 and the value of n is 10. In certain embodiments, the value of m is 12 and the value of n is 12.

In certain embodiments of the invention, the quaternary ammonium compound has a chemical structure comprising:

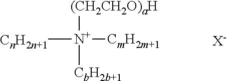

wherein the value of m is at least 1 and at most 20, the value of n is at least 1 and at most 20, the value of a is at least 1 and at most 5, the value of b is at least 1 and at most 5, and X⁻ is an anion selected from the group consisting of borate, chloride, carbonate, bicarbonate, bromide, iodide, formate, acetate, propionate, acetate, propionate, and other alkyl carboxylates. In certain embodiments, the value of m is at least 8 and at most 14. In certain embodiments, the value of n is at least 8 and at most 14. In certain embodiments, the value of a is 1, the value of b is 1. In certain embodiments X⁻ is borate, chloride, propionate, carbonate, or bicarbonate. In certain embodiments, the value of m is 10 and the value of n is 10. In certain embodiments, the value of m is 12 and the value of n is 12.

In various embodiments of the invention, the quaternary ammonium compound may be didecyldimethylammonium carbonate or bicarbonate, didecyldimethylammonium chloride, lauryl trimethyl ammonium chloride, coco bis(2-hydroxyethyl)methylammonium chloride, didodecyldimethylammonium chloride, and didodecyldimethylammonium carbonate or bicarbonate, N,N-Didecyl-N-methyl-poly(oxyethyl) ammonium propionate, didecyl bis(hydroxyethyl) ammonium borate.

The quaternary ammonium compounds may be added directly to a treating solution, or may be formulated into a concentrate, which can be later diluted to prepare a final treating composition. In certain embodiments, the quaternary ammonium compound is present in the wood preservative treating composition in an amount between about 0.01% (wt/wt) to about 0.5% (wt/wt); or between about 0.01% (wt/wt) to about 0.2% (wt/wt); or between about 0.03% (wt/wt) to about 0.15% (wt/wt); or between about 0.05% (wt/wt) to about 0.10% (wt/wt); or between about 0.1% (wt/wt) to about 0.2% (wt/wt).

In certain embodiments of the invention, the nonionic surfactant compound is an aromatic ethoxylate, or an alkylphenol ethoxylate, such as an octylphenol ethoxylate, a nonylphenol ethoxylate, a dinonylphenol ethoxylate, a tristriphenol ethoxylate, or a dodecylphenol ethoxylate. The degree of ethoxylation (the moles of ethylene oxide “EO”) can vary from 1 to 500. The preferred moles of EO is between 8 and 100; or between 15 and 50; or between 20 and 40.

In certain embodiments of the invention, the nonionic surfactant is an alcohol ethoxylate. The alcohol can be a primary or a secondary alcohol, branched or linear or mixed branched and linear. The alcohol carbon chain length can vary from 2 to 50 carbons. Non-limiting examples of alcohols are branched isotridecyl alcohol, branched isodecyl alcohol, oleyl alcohol, allyl alcohol, lauryl alcohol, myristyl alcohol, cetyl alcohol, stearyl alcohol, ceryl alcohol, etc. The degree of ethoxylation (the moles of ethylene oxide “EO”) can vary from 1 to 500. The preferred moles of EO is between 8 and 100; or between 15 and 50; or between 20 and 40.

In certain embodiments of the invention, the nonionic surfactant compound is a block copolymer of ethylene oxide (EO) and propylene oxide (PO), or a branched/linear alcohol alkoxylate with EO/PO or phenol alkoxylate with EO/PO copolymer.

In certain embodiments of the invention, the nonionic surfactant compound is a fatty amide, an alkanolamide or ethylene bisamide. Examples of fatty acids used for making fatty amides or alkanolamides include, but are not limited to, oleic, erucic, coconut, linoleic, lauric, stearic, cerotic, capric, caprylic, and palmitic acids. Examples of alkanolamides include, but are not limited to, monoethanolamides, diethanolamides, and diethanolamides.

In certain embodiments of the invention, the nonionic surfactant compound is a fatty ester, glycerol ester, glycol ester, alcohol ester, ethoxylated fatty acids, glycol and polyethylene glycol (PEG) esters, ethoxylated fatty oil, ethoxylated sorbitan esters, ethoxylated castor oil, sorbitol esters, and ethoxylated sorbitol esters.

In certain embodiments of the invention, the nonionic surfactants are fatty acid polyglycol esters, fattyamide, cocamide DEA, cocamide MEA, secondary alcohol ethoxylates, alkylphenol ethoxylates, alkylaryl polyglycol ethers, fatty alcohol polyglycol ethers, propylene oxide-ethylene oxide, alkyl polyethers, sorbitan esters, for example sorbitan fatty acid esters, or polyoxyethylene sorbitan fatty acid esters. The nonionic surfactant in the wood preservative treating composition has a HLB value greater than 10, 11, 12, 13, 14 or 15; or a HLB value between 10 and 40; or between 10 and 30; or between 10 and 20; or between 14 and 18.

The nonionic surfactants may be added directly to a treating solution, or may be formulated into a concentrate, which can be later diluted to prepare a final treating composition. In certain embodiments, the nonionic surfactant is present in the wood preservative treating composition in an amount between about 0.01% (wt/wt) to about 5.0% (wt/wt); or between about 0.01% (wt/wt) to about 1.0% (wt/wt); or between about 0.05% (wt/wt) to about 0.25% (wt/wt); or between about 0.05% (wt/wt) to about 0.15% (wt/wt); or between about 0.05% (wt/wt) to about 0.1% (wt/wt).

Triazoles:

Triazoles of the wood preservative formulations of the invention include, but are not limited to epoxiconazole, triadimenol, propiconazole, prothioconazole, metconazole, cyproconazole, tebuconazole, flusilazole, penflufen, paclobutrazol, fluconazole, isavuconazole, itraconazole, voriconazole, pramiconazole, ravuconazole, and posaconazole.

Surfactants and Emulsifiers:

Surfactants and emulsifiers may be combined with the triazoles of the formulations of the invention to increase their solubility. Examples of surfactants and emulsifiers that may be used include, but are not limited to, ionic and/or nonionic surfactants and emulsifiers. These include, but are not limited to, for example calcium alkylarylsulfonates such as calcium dodecylbenzenesulfonate, or nonionic emulsifiers such as fatty acid polyglycol esters, fattyamide, cocamide DEA, cocamide MEA, secondary alcohol ethoxylates, alkylphenol ethoxylates, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Copper and Copper Compounds:

The solubilized copper, or solubilized copper compounds, or solid copper, or solid copper compounds of the wood preservative formulations of the invention are prepared from: but are not limited to copper metal, cuprous oxide (a source of copper (I) ions), cupric oxide (a source of copper (II) ions), copper hydroxide, copper carbonate, basic copper carbonate, copper oxychloride, copper 8-hydroxyquinolate, copper dimethyldithiocarbamate, copper omadine, copper borate, copper residues (copper metal byproducts) or any suitable copper source.

Copper Dispersing or Solubilizing Agents:

Copper compositions disclosed in the current invention can be either particulate copper dispersions or soluble copper solutions. In the case of particulate copper dispersions, copper compounds are dispersed with the aid of polymeric dispersant(s). In the case of soluble copper solution, copper compounds may be solubilized by contacting them with a solubilizing agent. Examples of solubilizing agents include, but are not limited to, alkanolamines, such as, for example, monoethanolamine, ethanolamine, diethanolamine, triethanolamine and ammonia.

Copper Penetration Passing Rate:

As used herein, the term “copper penetration passing rate” means the percentage of wood borings taken from treated wood that meets or exceeds the penetration specifications, i.e. penetration depth and/or percent of sapwood, as described in American Wood Protection Association Standard T1-15 “Use Category System: Processing and Treatment Standard” (2015) which is incorporated herein by reference in its entirety. For example, if 20 borings from a sample of treated wood product are taken, and 10 of the borings meet or exceed the penetration specifications, then the copper penetration passing rate for the treated wood product is 50%.

Total Copper Azole Concentration:

As used herein, the term “total copper azole concentration” refers to the total combined weight percentage (wt/wt) of copper and azole in the formulation. In certain embodiments, the total copper azole concentration is present in the wood preservative treating composition in an amount between about 0.01% (wt/wt) to about 5.0% (wt/wt); or between about 0.05% (wt/wt) to about 2.0% (wt/wt); or between about 0.1% (wt/wt) to about 1.0% (wt/wt); or between about 0.2% (wt/wt) to about 0.8% (wt/wt); or between about 0.5% (wt/wt) to about 1.5% (wt/wt).

In certain embodiments, the treatment solution containing a quaternary ammonium compound, or a nonionic surfactant, may be used to treat wood for preservation. In these embodiments, the wood may be in nominal size of 1″×6″, 2″×4″, 2″×6″, 2″×8″, 2″×10″, 2″×12″, 2″×14″pieces of wood lumber, or nominal size of 4″×4″, 4″×6″, 6″×6″ wood timber or other nominal size of round wood timber and lumber. The types of wood that may be preserved with the treatment solution include, but are not limited to, Douglas fir, Hem-fir, Nordic pine, Scotts pine, Norway spruce, Sitka spruce, southern yellow pine, incised Douglas fir, Spruce pine fir, red pine, and ponderosa pine.

In certain embodiments, the wood to be treated with the treatment solution is placed into a pressure chamber. The wood may then undergo a vacuum/pressure treating cycle in which air is evacuated from (and the pressure lowered in) the pressure chamber during an initial vacuum stage, the pressure raised and the wood treated with the treatment solution during a pressure treatment stage, and then the pressure lowered and the air evacuated from the chamber during a final vacuum stage.

In certain embodiments, the pressure in the chamber during the initial vacuum stage may be between about 10 and 29 inches Hg, the pressure in the chamber during the pressure treating stage may be between about 100-200 psi, or between about 145-200 psi, or between about 145-180 psi, and the final vacuum stage may be between about 10 and 29 inches Hg. The initial vacuum stage may last between about 5 minutes and about 60 minutes, the pressure treating stage may last between about 5 minutes and 300 minutes, or between about 60-300 minutes, or between about 90-240 minutes, and the final vacuum stage may last between about 5 minutes and about 60 minutes, or between about 15 minutes to about 45 minutes.

In certain embodiments, the treating solutions comprising the current compositions may be heated to elevated temperatures. The temperature ranges from ambient to 150° F. or from ambient to about 120° F. The ambient temperature depends upon the temperature of the makeup water used to make up the treating solutions and the local environmental conditions.

The following Examples are only illustrative. It will be readily seen by one of ordinary skill in the art that the present invention fulfills the objectives set forth above. After reading the foregoing specification, one of ordinary skill will be able to effect various changes, substitutions of equivalents, and various other embodiments of the invention as broadly disclosed therein. It is therefore intended that the protection granted herein be limited only by the definition contained in the appended claims and equivalents thereof.

### EXAMPLES

### Example 1

Samples of wood were pressure treated with copper triazole preservation solutions containing a quaternary ammonium compound (a “quat”), as well as copper triazole preservation solutions that did not contain a quat.

The samples were all subjected to the same vacuum/pressure treatment cycle, consisting of: a) an initial vacuum treatment of between about 24 and 29 inches Hg for 30 minutes; b) a pressure treatment of between about 150-165 psi for 240 minutes; and c) a final vacuum treatment of between about 24 and 29 inches Hg for 30 minutes.

The effects of these treatments were as follows:

TABLE 1
Effects on Copper Penetration of Adding Quaternary
Ammonium Compounds to Copper Azole Formulations

| | | Total | | | | | Copper |
|---|---|---|---|---|---|---|---|
| | Treating | Cu/Azole | Quat | Quat | Wood | Nominal | Penetration |
| # | Solution | Conc'n¹ | Type | Conc'n | Type | Size (in.) | Passing Rate |
| 1 | Copper Amine + | 1.1% | N/A | 0.0% | Incised | 2 × 4 | 40% |
| | Azoles | | | | Douglas Fir | | |
| 2 | Copper Amine + | 1.1% | N/A | 0.0% | Incised | 4 × 4 | 50% |
| | Azoles | | | | Douglas Fir | | |
| 3 | Copper Amine + | 1.1% | Quat #1 | 0.1% | Incised | 2 × 4 | 80% |
| | Azoles | | | | Douglas Fir | | |
| 4 | Copper Amine + | 1.1% | Quat #1 | 0.1% | Incised | 4 × 4 | 80% |
| | Azoles | | | | Douglas Fir | | |
| 5 | Copper Amine + | 1.1% | Quat #l | 0.1% | Incised | 4 × 4 | 80% |
| | Azoles | | | | Douglas Fir | | |
| 6 | Copper Amine + | 1.1% | Quat #l | 0.1% | Incised | 2 × 4 | 85% |
| | Azoles | | | | Douglas Fir | | |
| 7 | Copper Amine + | 1.1% | Quat #2 | 0.1% | Incised | 2 × 4 | 65% |
| | Azoles | | | | Douglas Fir | | |
| 8 | Copper Amine + | 1.1% | Quat #3 | 0.1% | Incised | 2 × 4 | 80% |
| | Azoles | | | | Douglas Fir | | |
| 9 | Copper Amine + | 1.1% | Quat #3 | 0.1% | Incised | 2 × 4 | 80% |
| | Azoles | | | | Douglas Fir | | |
| 10 | Copper Amine + | 1.1% | Quat #4 | 0.1% | Incised | 2 × 4 | 80% |
| | Azoles | | | | Douglas Fir | | |
| 11 | Copper Amine + | 1.1% | Quat #4 | 0.1% | Incised | 4 × 4 | 85% |
| | Azoles | | | | Douglas Fir | | |
| 12 | Copper Amine + | 1.1% | Quat #4 | 0.1% | Incised | 2 × 4 | 80% |
| | Azoles | | | | Douglas Fir | | |

¹The abbreviation “Conc'n” means Concentration

In the above table, the copper penetration passing rate is defined as the percentage of treated samples that met the copper penetration conformance standard defined in the T1-15 Processing and Treatment Standard of the American Wood Protection Association (AWPA). “Quat #1” is the quaternary ammonium compound didecyldimethylammonium bicarbonate/carbonate, “Quat #2” stands for lauryl trimethyl ammonium chloride, “Quat #3” is coco bis (2-hydroxyethyl)methyl ammonium chloride, and “Quat #4” stands for didecyldimethylammonium chloride.

### Example 2

Samples of wood were pressure treated with copper triazole preservation solutions containing a quaternary ammonium compound (a “quat”), as well as copper triazole preservation solutions that did not contain a quat.

The samples were all subjected to the same vacuum/pressure treatment cycle, consisting of: a) an initial vacuum treatment of between about 24 and 29 inches Hg for 15 minutes; b) a pressure treatment of between about 150-165 psi for 45 minutes; and c) a final vacuum treatment of between about 24 and 29 inches Hg for 30 minutes.

The effects of these treatments were as follows:

TABLE 2
Effects on Copper Penetration of Adding Quaternary
Ammonium Compounds to Copper Azole Formulations

| | | Total | | | | | Copper |
|---|---|---|---|---|---|---|---|
| | Treating | Cu/Azole | Quat | Quat | Wood | Nominal | Penetration |
| # | Solution | Conc'n | Type | Conc'n | Type | Size (in.) | Passing Rate |
| 1 | Copper Amine + | 1.0% | N/A | 0.0% | Hem-Fir | 2 × 6 | 45% |
| | Azoles | | | | | | |
| 2 | Copper Amine + | 1.0% | N/A | 0.0% | Hem-Fir | 2 × 6 | 40% |
| | Azoles | | | | | | |
| 3 | Copper Amine + | 1.0% | Quat #4 | 0.1% | Hem-Fir | 2 × 6 | 90% |
| | Azoles | | | | | | |
| 4 | Copper Amine + | 1.0% | Quat #4 | 0.1% | Hem-Fir | 2 × 6 | 95% |
| | Azoles | | | | | | |
| 5 | Copper Amine + | 1.0% | Quat #3 | 0.1% | Hem-Fir | 2 × 6 | 95% |
| | Azoles | | | | | | |
| 6 | Copper Amine + | 1.0% | Quat #3 | 0.1% | Hem-Fir | 2 × 6 | 80% |
| | Azoles | | | | | | |
| 7 | Copper Amine + | 1.0% | Quat #2 | 0.1% | Incised | 2 × 6 | 75% |
| | Azoles | | | | Douglas Fir | | |
| 8 | Copper Amine + | 1.0% | Quat #2 | 0.1% | Incised | 2 × 6 | 80% |
| | Azoles | | | | Douglas Fir | | |

In the above table, the copper penetration passing rate is defined as the percentage of treated samples that met the copper penetration conformance standard defined in the T1-15 Processing and Treatment Standard of by the American Wood Protection Association (AWPA). “Quat #2” is lauryl trimethyl ammonium chloride, “Quat #3” is coco bis(2-hydroxyethyl)methylammonium chloride, and “Quat #4” is didecyldimethylammonium chloride.

### Example 3

Samples of wood were pressure treated with copper triazole preservation solutions containing a quaternary ammonium compound (a “quat”), as well as copper triazole preservation solutions that did not contain a quat.

The samples were all subjected to the same vacuum/pressure treatment cycle, consisting of: a) an initial vacuum treatment of between about 24 and 29 inches Hg for 20 minutes; b) a pressure treatment of between about 150-165 psi for 60 minutes; and c) a final vacuum treatment of between about 24 and 29 inches Hg for 20 minutes.

The effects of these treatments were as follows:

TABLE 3
Effects on Copper Penetration of Adding Quaternary
Ammonium Compounds to Copper Azole Formulations

| | | Total | | | | | Copper |
|---|---|---|---|---|---|---|---|
| | Treating | Cu/Azole | Quat | Quat | Wood | Nominal | Penetration |
| # | Solution | Conc'n | Type | Conc'n | Type | Size (in.) | Passing Rate |
| 1 | Copper Amine + | 1.0% | N/A | 0.0% | Douglas Fir | 4 × 4 | 69% |
| | Azoles | | | | | | |
| 2 | Copper Amine + | 1.0% | Quat #4 | 0.1% | Douglas Fir | 4 × 4 | 81% |
| | Azoles | | | | | | |
| 3 | Copper Amine + | 1.0% | Quat #3 | 0.1% | Douglas Fir | 4 × 4 | 81% |
| | Azoles | | | | | | |
| 4 | Copper Amine + | 1.0% | Quat #5 | 0.1% | Douglas Fir | 4 × 4 | 72% |
| | Azoles | | | | | | |

In the above table, the copper penetration passing rate is defined as the percentage of treated samples that met the copper penetration conformance standard defined in the 11-15 Processing and Treatment Standard of the American Wood Protection Association (AWPA). “Quat #3” is coco bis(2-hydroxyethyl)methylammonium chloride, “Quat #4” didecyldimethylammonium chloride, and “Quat #5” is didodecyldimethylammonium chloride.

### Example 4

A soluble copper triazole preservative solution that did not contain a penetrating enhancer was used as a reference to pressure treat Douglas fir (DF). Soluble copper triazole preservative solutions containing a penetration enhancer were used to treat the same stock of DF as the ones treated without an enhancer.

The DF samples were all subjected to the same vacuum/pressure treatment cycle, consisting of: a) an initial vacuum treatment of between about 24 and 29 inches Hg for 20 minutes; b) a pressure treatment of between about 150-165 psi for 60 minutes; and c) a final vacuum treatment of between about 24 and 29 inches Hg for 20 minutes.

The effects of these treatments were as follows:

TABLE 4
Effects on Copper Penetration of Adding Penetration
Enhancers (PEs) to Copper Azole Formulations

| | | Total | | | | | Copper |
|---|---|---|---|---|---|---|---|
| | Treating | Cu/Azole | Penetration | PE | Wood | Nominal | Penetration |
| # | Solution | Conc'n¹ | Enhancer | Conc'n | Type | Size (in.) | Passing Rate |
| 1 | Copper Amine + | 1.1% | N/A | 0.0% | Incised | 2 × 6 | 61% |
| | Azoles | | | | Douglas Fir | | |
| 2 | Copper Amine + | 1.1% | Surfactant | 0.1% | Incised | 2 × 6 | 94% |
| | Azoles | | #1 | | Douglas Fir | | |
| 3 | Copper Amine + | 1.1% | Surfactant | 0.2% | Incised | 2 × 6 | 83% |
| | Azoles | | #1 | | Douglas Fir | | |
| 4 | Copper Amine + | 1.1% | Surfactant | 0.1% | Incised | 2 × 6 | 94% |
| | Azoles | | #2 | | Douglas Fir | | |
| 5 | Copper Amine + | 1.1% | Surfactant | 0.2% | Incised | 2 × 6 | 100% |
| | Azoles | | #2 | | Douglas Fir | | |

¹The abbreviation “Conc'n” means Concentration

In the above table, the copper penetration passing rate is defined as the percentage of treated samples that met the copper penetration conformance standard defined in the T1-15 Processing and Treatment Standard of the American Wood Protection Association (AWPA). Surfactant #1″ is a nonylphenol ethoxylate (or nonyl phenoxypolyethoxylethanol) with an HLB of 15.0-19.0. “Surfactant #2” is an ethoxylated secondary alcohol with carbon chain length of 12-14 and has an HLB of 14.0-18.0.

### Example 5

Samples of wood were pressure treated with copper triazole preservation solutions containing a penetration enhancer, as well as copper triazole preservation solutions that did not contain a penetration enhancer.

The samples were all subjected to the same vacuum/pressure treatment cycle, consisting of: a) an initial vacuum treatment of between about 24 and 29 inches Hg for 20 minutes; b) a pressure treatment of between about 150-165 psi for 90 minutes; and c) a final vacuum treatment of between about 24 and 29 inches Hg for 20 minutes.

The effects of these treatments were as follows:

TABLE 5
Effects on Copper Penetration of Adding Penetration
Enhancer (PE) to Copper Azole formulations

| | | Total | | | | | Copper |
|---|---|---|---|---|---|---|---|
| | Treating | Cu/Azole | PE | PE | Wood | Nominal | Penetration |
| # | Solution | Conc'n | Type | Conc'n | Type | Size (in.) | Passing Rate |
| 1 | Copper Amine + | 1.0% | N/A | 0.0% | Douglas-Fir | 2 × 6 | 61% |
| | Azoles | | | | | | |
| 2 | Copper Amine + | 1.0% | Surfactant | 0.1% | Douglas-Fir | 2 × 6 | 83% |
| | Azoles | | #2 | | | | |

In the above table, the copper penetration passing rate is defined as the percentage of treated samples that met the copper penetration conformance standard defined in the T1-15 Processing and Treatment Standard of by the American Wood Protection Association (AWPA). Surfactant #2″ is an ethoxylated secondary alcohol with carbon chain length of 12-14 and has a HLB of 14.0-18.0.

### Example 6

DF wood is pressure treated with copper triazole preservation solution containing a penetration enhancer, as well as copper triazole preservation solutions without containing a penetration enhancer. The penetration enhancer is an alkylphenol ethoxylate surfactant with a HLB value of 12.0-15.0. The samples are all subjected to the same vacuum/pressure treatment cycle, consisting of: a) an initial vacuum treatment of between about 24 and 29 inches Hg for 15 minutes; b) a pressure treatment of between about 150 psi for 150 minutes; and c) a final vacuum treatment of between about 24 and 29 inches Hg for 30 minutes. After treatment, the copper treating solution containing the penetration enhancer results in significantly higher copper penetration rate than the one without the penetration enhancer.

### Example 7

Hem fir wood is pressure treated with copper triazole preservation solution containing a penetration enhancer, as well as copper triazole preservation solutions without containing a penetration enhancer. The penetration enhancer is an alkylphenol ethoxylate surfactant with a HLB value of 14.0-18.0. The samples are all subjected to the same vacuum/pressure treatment cycle, consisting of: a) an initial vacuum treatment of between about 24 and 29 inches Hg for 15 minutes; b) a pressure treatment of between about 150 psi for 150 minutes; and c) a final vacuum treatment of between about 24 and 29 inches Hg for 30 minutes. After treatment, the copper treating solution containing the penetration enhancer results in significantly higher copper penetration rate than the one without the penetration enhancer.

### Example 8

A set of refractory southern pine wood is pressure treated with copper triazole preservation solution containing a penetration enhancer, as well as copper triazole preservation solution without containing a penetration enhancer. The penetration enhancer is a secondary alcohol ethoxylate surfactant with a HLB value of 16.0-18.0. The samples are all subjected to the same vacuum/pressure treatment cycle, consisting of: a) an initial vacuum treatment of between about 24 and 29 inches Hg for 5 minutes; b) a pressure treatment of between about 150 psi for 15 minutes; and c) a final vacuum treatment of between about 24 and 29 inches Hg for 30 minutes. After treatment, the copper treating solution containing the penetration enhancer results in significantly higher copper penetration rate than the one without the penetration enhancer.

### Example 9

A set of refractory red pine wood is pressure treated with copper triazole preservation solution containing a penetration enhancer, as well as copper triazole preservation solution without containing a penetration enhancer. The penetration enhancer is a secondary alcohol ethoxylate surfactant with a HLB value of 15.0-19.0. The samples are all subjected to the same vacuum/pressure treatment cycle, consisting of: a) an initial vacuum treatment of between about 24 and 29 inches Hg for 15 minutes; b) a pressure treatment of between about 150 psi for 60 minutes; and c) a final vacuum treatment of between about 24 and 29 inches Hg for 30 minutes. After treatment, the copper treating solution containing the penetration enhancer results in significantly higher copper penetration rate than the one without the penetration enhancer.

1-129. (canceled)

130. A method for increasing penetration of a wood preservative composition into a wood product, or decreasing the processing time of a wood preservative composition into a wood product, the method comprising contacting a wood preservative composition with a wood product, wherein said wood preservative composition comprises:
a. copper compound;
b. a triazole; and
c. a penetration enhancer comprising a nonionic surfactant;
wherein said wood preservative composition penetrates said wood product to a greater degree, or in less time, than said wood preservative composition lacking a penetration enhancer.

131. The method of claim 130, wherein said nonionic surfactant is selected from the group consisting of ethoxylates, alkylphenol ethoxylates, octylphenol ethoxylates, nonylphenol ethoxylates, dinonylphenol ethoxylates, phenol ethoxylates and dodecylphenol ethoxylates.

132. The method of claim 130, wherein said copper compound is selected from the group consisting of copper metal, cuprous oxide, cupric oxide, copper hydroxide, copper carbonate, basic copper carbonate, copper oxychloride, copper 8-hydroxyquinolate, copper dimethyldithiocarbamate, copper omadine, and copper borate.

133. The method of claim 132, wherein said copper compound is solubilized in the presence of an amine.

134. The method of claim 133, wherein said amine is selected from the group consisting of alkanolamine, monoethanolamine, ethylenediamine, diethanolamine, triethanolamine and ammonia.

135. The method of claim 133, wherein said solubilized copper compound is prepared from copper metal, copper hydroxide, cuprous oxide, cupric oxide, copper carbonate or basic copper carbonate.

136. The method of claim 130, wherein said triazole is tebuconazole, or propiconazole, or cyproconaozle, or penflufen.

137. The method of claim 130, wherein said penetration enhancer is present in said wood preservative composition in an amount between about 0.01% (wt/wt) to about 0.5% (wt/wt).

138. The method of claim 130, wherein said penetration enhancer is present in said wood preservative composition in an amount between about 0.01% (wt/wt) to about 0.2% (wt/wt).

139. The method of claim 130, wherein said penetration enhancer is present in said wood preservative composition in an amount between about 0.03% (wt/wt) to about 0.15% (wt/wt).

140. The method of claim 130, wherein said penetration enhancer is present in said wood preservative composition in an amount between about 0.05% (wt/wt) to about 0.1% (wt/wt).

141. The method of claim 130, wherein said penetration enhancer is present in said wood preservative composition in an amount between about 0.1% (wt/wt) to about 0.2% (wt/wt).

142. The method of claim 130, wherein the copper penetration passing rate in said wood product contacted with said wood preservative composition is at least about 15% greater than the copper penetration passing rate of said wood product treated with said wood preservative composition lacking said penetration enhancer.

143. The method of claim 130, wherein the copper penetration passing rate in said wood product contacted with said wood preservative composition is at least about 20% greater than the copper penetration passing rate of said wood product treated with said wood preservative composition lacking said penetration enhancer.

144. The method of claim 130, wherein the copper penetration passing rate in said wood product contacted with said wood preservative composition is at least about 25% greater than the copper penetration passing rate of said wood product treated with said wood preservative composition lacking said penetration enhancer.

145. The method of claim 130, wherein the copper penetration passing rate in said wood product contacted with said wood preservative composition is at least about 30% greater than the copper penetration passing rate of said wood product treated with said wood preservative composition lacking said penetration enhancer.

146. The method of claim 130, wherein said copper penetration passing rate in said wood product contacted with said wood preservative composition is at least about 35% greater than the copper penetration passing rate of said wood product treated with said wood preservative composition lacking said penetration enhancer.

147. The method of claim 130, wherein said copper penetration passing rate in said wood product contacted with said wood preservative composition is at least about 40% greater than the copper penetration passing rate of said wood product treated with said wood preservative composition lacking said penetration enhancer.

148. The method of claim 130, wherein said copper penetration passing rate in said wood product contacted with said wood preservative composition is at least about 45% greater than the copper penetration passing rate of said wood product treated with said wood preservative composition lacking said penetration enhancer.

149. The method of claim 130, wherein said copper penetration passing rate in said wood product contacted with said wood preservative composition is at least about 50% greater than the copper penetration passing rate of said wood product treated with said wood preservative composition lacking said penetration enhancer.

150. The method of claim 130, wherein the copper penetration passing rate in said wood product contacted with said wood preservative composition is at least about 55% greater than the copper penetration passing rate of said wood product treated with said wood preservative composition lacking said penetration enhancer.

151. The method of claim 130, wherein said wood product is a wood species selected from the group consisting of Douglas fir, Hem-fir, Nordic pine, Scotts pine, Norway spruce, Sitka spruce, southern yellow pine, incised Douglas fir, and incised Hem-fir.

152. A wood product treated by the method of claim 130.
